(19) United States
(12) Patent Application Publication
(10) Pub. No.: US 2021/0244957 A1
(43) Pub. Date: Aug. 12, 2021

(54) Primates Depression Treatment Apparatus and Driving Method Thereof

(71) Applicant: Whi Young KIM, Busan (KR)

(72) Inventor: Whi Young KIM, Busan (KR)

(21) Appl. No.: 16/802,594

(22) Filed: Feb. 27, 2020

(30) Foreign Application Priority Data
Feb. 7, 2020 (KR) 10-2020-0014985

(51) Int. Cl. A61N 2/00; A61N 2/02; G16H 20/40; G16H 40/63; G16H 50/20

(52) U.S. Cl. CPC A61N 2/006; A61N 2/02; G16H 50/20; G16H 40/63; G16H 20/40

(57) ABSTRACT

A primates depression treatment apparatus according to an embodiment of the present invention include: a magnetic stimulation coil unit outputting magnetic stimulation having different characteristics corresponding to an asymmetry degree of a brain; and a controller determining an asymmetry degree between a left hemisphere and a right hemisphere on the basis of brain wave measurement data of the brain, and controlling the magnetic stimulation coil unit to output the magnetic stimulation having difference characteristics on the basis of control data that are determined in accordance with the determination result.

### BACKGROUND OF THE INVENTION

### Field of the Invention

The present invention relates to a primates depression treatment apparatus and a driving method thereof and, more particularly, to a primates depression treatment apparatus that performs magnetic stimulation treatment in accordance with the degree of asymmetry between the left hemisphere and the right hemisphere of a brain on the basis of brain wave measurement data obtained by measuring brain waves, for example, through an Electroencephalography (EEG) sensor, and a method of driving the primates depression treatment apparatus.

### Description of the Related Art

As the mechanisms causing depression, there are genetic factors, a monoamine deficit hypothesis, stress, malfunction of a hypothalamus-pituitary gland-adrenal cortex axis, etc., and a variation of a promoter region (serotonin-transporter-linked polymorphic region, 5-HTTLPR), stress, etc. may be factors causing depression. A noradrenaline-serotonin system spreads to all parts in the deep part of a brain and influences the main functions of the brain, the activity of neurotransmitters of norepinephrine, serotonin, and monoamine is increased, and the activity of monoamine oxidase that decomposes norepinephrine and serotonin in a brain increases 30%.

Recognition is made by the cerebral cortex and the amygdala of the limbic system, a signal is transmitted to the hypothalamus, the hypothalamus receiving a stress signal simulates the receptor of the pituitary gland by secreting adrenocorticotropic hormone (ACTH) and corticotrophin releasing hormone (CRH), the secreted adrenocorticotropic hormone promotes secretion of cortisol from the adrenal cortex, and the cortisol secreted in the blood reduces secretion of the CRH and the ACTH to the hypothalamus and the pituitary gland through the negative feedback mechanism, so homeostasis is maintained, that is, cortisol and CRH are involved with depression.

In depression, plasma cortisol, cerebrospinal fluid, and an increase of CRH in the limbic system are observed, secretion of CRH by dexamethasone is suppressed, improvement of symptoms by antidepressant is related to recovery of the functions of the hypothalamus-pituitary gland-adrenal cortex and is greatly associated with neuroendocrine, an increase of cortisol suppresses propagation of nerve cells, and the size of the hippocampus decreases.

Mitochondria produces ATP (adenosine triphosphate), which is the main energy source for cells, through aerobic oxidation and the main function is performed by an electron transport enzyme composite I-V, two electron carriers, coenzyme Q10, and cytochrome C. Mitochondria has an important role in cell life activity such as adjustment of apoptosis and calcium ions and is related with congenital genetic diseases in DNA, aging, diabetes mellitus type 2, neurodegenerative diseases, cardiac disease, cancer, etc., and synthesis, secretion, and resorption of neurotransmitters such as serotonin and norepinephrine require a lot of energy, and energy production in cells is in association with depression.

In depression, deterioration of activity of the right dorsal prefrontal lobe and the dorsal anterior cingulate cortex (Brodman area, hereafter, ‘BA’) and an increase of activity of the limbic system and the frontal dorsal anterior cingulate cortex (BA25) appear, activity of the interior frontal lobe and the right anterior cingulate cortex (BA 32) increases, sadness inducement is in connection with deterioration of activity of the frontal lobe cortex and activation of the limbic system and the peripheral limbic system, the cortex thickness of the lateral orbitofrontal cortex is small as 12˜15%, the density of the centrum and the neuroglia cells decrease 16˜30%, large neuroglia cells are less by 22˜37%, small neuroglia cells are much by 6˜27%, and the volume reduction of the brain in depression is caused by atrophy rather than a loss of neuroglia cells. Further, the volume of the frontal lobe is small as 7%, the volume of the subgenual prefrontal cortex is 44%, the volume of the orbitofrontal cortex is small by as 32%, that is, the volume of the frontal lobe cortex decreases, the activity of the left dorsal prefrontal cortex and the anterior cingulate cortex is deteriorated and the activity of the ventral prefrontal lobe in a dormancy state and a simple dormancy state, serotonin is important monoamine related to a metal disease and pathology, and serotonin decreases due to suicide relevance.

In the related art, depression is cured by medicines in most cases and the medicines are limitedly applied only when depression is diagnosed, so there is a problem that generality and usefulness are low.

### DOCUMENTS OF RELATED ART

(Patent Document 0001) Korean Patent No. 10-2068401 (2020 Jan. 14)

(Patent Document 0002) Korean Patent Application Publication No. 10-2019-0117385 (2019 Oct. 16)

### SUMMARY OF THE INVENTION

An embodiment of the present invention provides a primates depression treatment apparatus that performs magnetic stimulation treatment in accordance with the degree of asymmetry between the left hemisphere and the right hemisphere of a brain on the basis of brain wave measurement data obtained by measuring brain waves, for example, through an Electroencephalography (EEG) sensor, and a method of driving the primates depression treatment apparatus.

A primates depression treatment apparatus according to an embodiment of the present invention includes: a magnetic stimulation coil unit outputting magnetic stimulation having different characteristics corresponding to an asymmetry degree of a brain; and a controller determining an asymmetry degree between a left hemisphere and a right hemisphere on the basis of brain wave measurement data of the brain, and controlling the magnetic stimulation coil unit to output the magnetic stimulation having difference characteristics on the basis of control data that are determined in accordance with the determination result.

The controller may create quantified brain wave imbalance data of depression on the basis of the brain wave measurement data, may classify the created brain wave imbalance data in accordance with a state of the brain, and may output the control data on the basis of the classified brain wave imbalance data.

The controller may perform classification according to the state of the brain on the basis of a first analysis result of an α-wave, a β-wave, a γ-wave, a δ-wave, and a θ-wave on the basis of the brain wave measurement data and a second analysis result about sizes of the left hemisphere and the right hemisphere.

The controller may apply a Support Vector Machine (SVM) model to classify the brain imbalance data according to the state of the brain.

The controller may perform wired or wireless communication for providing the control data to the magnetic stimulation coil unit when the controller is disposed in a device separated from the magnetic stimulation coil unit.

A method of driving a primates depression treatment apparatus according to an embodiment of the present invention includes: outputting magnetic stimulation having different characteristics corresponding to an asymmetry degree of a brain; and determining an asymmetry degree between a left hemisphere and a right hemisphere on the basis of brain wave measurement data of the brain, and controlling the magnetic stimulation coil unit to output the magnetic stimulation having difference characteristics on the basis of control data that are determined in accordance with the determination result, by means of a controller.

The method may further include: creating quantified brain wave imbalance data of depression on the basis of the brain wave measurement data; classifying the created brain wave imbalance data in accordance with a state of the brain; and outputting the control data on the basis of the classified brain wave imbalance data.

The classifying according to a state of the brain may perform classification according to the state of the brain on the basis of a first analysis result of an α-wave, a β-wave, a γ-wave, a δ-wave, and a θ-wave on the basis of the brain wave measurement data and a second analysis result about sizes of the left hemisphere and the right hemisphere.

The classifying according to a state of the brain may apply an SVM model to classify brain wave imbalance data according to the state of the brain.

The method may further include performing wired or wireless communication for providing the control data to the magnetic stimulation coil unit when the controller is disposed in a device separated from the magnetic stimulation coil unit.

A new antidepressant treatment method according to an embodiment of the present invention stimulates the cerebral cortex using a local magnetic field wave induced on the surface of the brain using magnetic stimulation, has a large possibility to be applied to treatment because it is a non-invasive operation method and has a local brain stimulation ability, and activates the prefrontal lobe, and has an anti-depressant effect on the basis of the fact that the function of the prefrontal lobe has an effect of depression treatment and there are a lot of studies about magnetic stimulation on the prefrontal lobe.

Further, the portable magnetic stimulation treatment apparatus according to an embodiment of the present invention can replace the stimulation coil in a detachable type and a superposition type, so it can not only provide simple nerve stimulation, but also convert nerve stimulation into nerve stimulation energy when a current pulse of magnetic stimulation is applied to a nerve and promotes nerve activity using the nerve stimulation generated by the bio nerve energy, thereby attenuating treatment such as depression and brain-related treatment and pain attenuation. Accordingly, the apparatus can be widely and efficiently used to prevent damage to the cranial nerves.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a diagram showing the entire configuration of a primates depression treatment apparatus according to an embodiment of the present invention;

FIG. 2 is a block diagram showing a process of processing brain wave measurement data;

FIG. 3 is a diagram showing a primates depression treatment process according to an embodiment of the present invention;

FIG. 4 is a diagram showing an analysis process of data;

FIG. 5 is a diagram showing a driving process of an analysis algorithm according to an embodiment of the present invention;

FIGS. 6 to 8 are diagrams showing a process for treatment, communication, control, and connection with an associated person; and

FIG. 9 is a flowchart showing a driving process of the primates depression treatment apparatus of FIG. 1 according to another embodiment of the present invention.

### DETAILED DESCRIPTION OF THE INVENTION

Hereinafter, embodiments of the present invention will be described in detail with reference to the accompanying drawings.

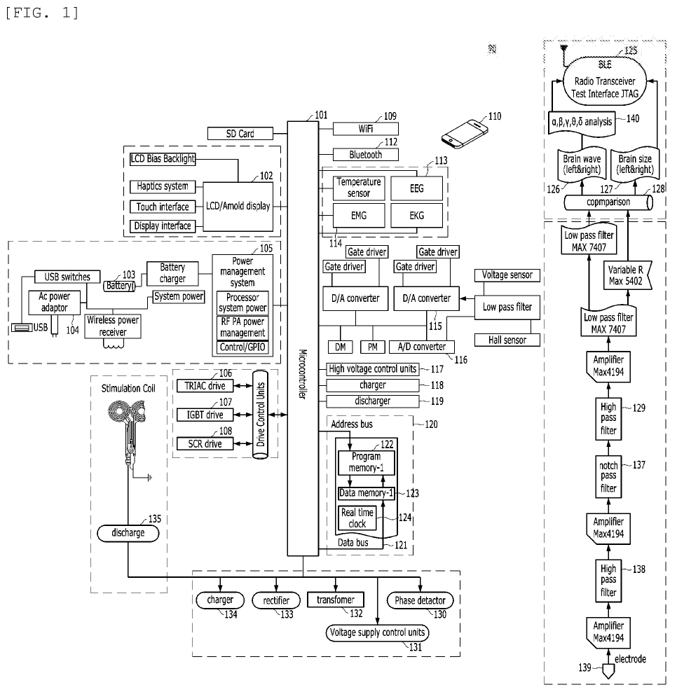

FIG. 1 is a diagram showing the entire configuration of a primates depression treatment apparatus according to an embodiment of the present invention.

As shown in FIG. 1, a primates depression treatment apparatus 90 according to an embodiment of the present invention includes some or all of a controller 101, a user interface 102, a power management unit 103˜105, a driving driver 106˜108, a communication unit 109˜112, a sensor unit (or sensing unit) 113, 114, a signal processor 115˜119, a memory unit 120˜124, and a depression treatment unit (or magnetic stimulation output unit) 125˜139.

The term ‘including some or all” means that the primates musculoskeletal treatment apparatus is configured as a separate apparatus without some components such as the depression treatment unit 125˜139 or some components such as the sensor unit 113, 114 are included in other components such as the controller 101, and it is exemplified in the following description that all of them are included to help sufficiently understand the present invention.

The controller 101 controls the general operations of various components shown in FIG. 1 of the primates musculoskeletal treatment apparatus 90. The controller 101 includes a microprocessor such as a CPU or an MPU and has an important role in the primates musculoskeletal treatment apparatus 90. For example, the controller 101 determines the degree of asymmetry between the left hemisphere and the right hemisphere of a brain on the basis of brain wave measurement data in accordance with an embodiment of the present invention and makes corresponding magnetic stimulation treatment be performed. If when the controller operates with a separate server for data analysis, the controller can provide brain wave measurement data to the server, receive corresponding treatment data or control data, and control the depression treatment unit 125˜139 on the basis of the data. Obviously, the depression treatment unit 125˜139 performs treatment using magnetic stimulation, so it may be a magnetic stimulation treatment unit.

The controller 101 according to an embodiment of the present invention can operate in various ways in accordance with how the primates depression treatment apparatus 90 is configured. Representatively, the operation may be different, depending on whether the controller 101 is integrated with the depression treatment unit 125˜139 or whether they are separately configured and operated in cooperation with each other. For example, when they are configured as separate devices and when the controller 101 is configured as one chip including a CPU and a memory, the controller 101 copies and loads operation programs according to an embodiment of the present invention to a memory and can perform magnetic stimulation treatment in accordance with the degree of asymmetry of the brain on the basis of brain wave measurement data by executing the operation program, so the controller is not specifically limited to any one type in an embodiment of the present invention. However, although it will be descried below in an embodiment of the present invention, data analysis is performed by the server because the amount of the data is large, it is possible to use applications (hereafter, app) of a smartphone, etc. for brain wave measurement, and the depression treatment apparatus performs magnetic stimulation treatment by receiving control data through short-range wireless communication employing Internet of Things (IoT).

The user interface 102, referring to FIG. 1, may include not only an LCD display window, but various interfaces controlling or related to the LCD display window. When a touch panel, etc. is included, a user interface is configured through the interface.

The power management unit 103˜105, when using a DC power having a built-in charging function, includes a battery power 103 and an AC adapter 104, etc., and may further include a power control manager 105. The AC adaptor 104 converts received commercial power into DC and the converted DC voltage can be stored in a battery. The power control manager 105 can measure the remaining amount of the battery and make it be displayed on the LCD display window, etc.

The driving driver 106˜108 can operate as a switching unit and can control output of magnetic stimulation that is output through a stimulation coil unit under control by the controller 101. In other words, the depression treatment unit 125˜139 according to an embodiment of the present invention may include a discharger that outputs high-frequency voltage to the magnetic coil and the driving driver 106˜108 can control the discharger. The driving driver 106˜108 may include a triac drive 106, an IGBT drive 107, and an SCR drive 108.

The communication unit 109˜112 may include a first communication unit 109 performing Wi-Fi communication with a cell phone 110 and a second communication unit 112 performing Bluetooth communication with peripheral devices. Obviously, the cell phone can perform Bluetooth communication too and may be implemented in various types, depending on the intention of a user.

The sensor unit (or sensing unit) 113, 114 includes various sensors such as an EEG sensor 113, an EKG (or ECG) sensor, and an EMG sensor 114, and may further include a temperature sensor. Various sensors configured in accordance with an embodiment of the present invention can be used for brain wave measurement.

The signal processor (or peripheral circuit) 115˜119 includes a D/A converter 115, an A/D converter 116, a high-voltage control signal unit 117, a charger 118, a discharge controller 119, etc. This is apparent to those skilled in the art, so it is no longer described.

The memory unit 120˜124 may include an address bus 120, a data bus 121, a program memory 122, a spare data memory 223, a clock generator 224, etc., in which the program memory 122 can perform an operation that calls out data stored in the data memory 223, etc. The clock generator 224 is in charge of real-time clocks. The clocks may be used to show start of data processing.

The depression treatment unit 125˜139 may include a magnetic stimulation coil unit or may be referred to as a magnetic stimulation coil unit and is an option of the function of an electroencephalography system, and particularly, reference numbers 130˜135 may indicate the internal configuration of the depression treatment apparatus. The depression treatment unit 125˜139 may include some or all of: a Bluetooth unit added to the sensor unit 113, 114; a third communication unit 125; a brain wave signal detector 126 that is an important part proposed in an embodiment of the present invention, detects a brain wave signal (left & right), and measures an abnormal signal to operate and control the treatment apparatus; a brain size signal processor 127 that is another important part, processes a brain size signal (left & right) to perform determination and control for operating the treatment apparatus when the sizes of the left and right brains are different; a comparer 128; a high-pass filter 138; an electrode 139; and a brain wave kind analyzer 140 that performs an important role for precisely increase the treatment effect by analyzing the kinds of brain waves on the basis of signals obtained from the brain wave signal detector 126. The term ‘include some or all’ is the same meaning as described above.

The primates depression treatment apparatus 90 according to an embodiment of the present invention is a stimulation pulse radiation and magnetic stimulation treatment apparatus that is used for the craniocervical nerves using magnetic pulses such as magnetic stimulation treatment for primates depression treatment, and has convenience is carrying and using. The primates depression treatment apparatus 90 converts nervous stimulation into nerve stimulation energy when applying a magnetic stimulation pulse to bio nerves and promotes nerve activity by stimulating nerves using the bio nerve energy in order to prevent damage to the cranial nerves by appeasing treatment such as treatment related to depression or the brain, and pain attenuation.

To this end, an embodiment of the present invention uses an International 10-20 system and an MCN (Modified Combinatorial Nomenclature) electrode disposition for analysis after brain wave measurement and brain wave extraction uses three kinds of signal processing methods to obtain desired information from raw data, which are preprocessing (ICA, Band Pass Filtering, Notch Filtering), feature extraction (PCA, power spectrum, autoregressive model), and classification (Hidden Markov Model, Kalman Filter Model, Multi-layer Perceptron Algorithm). As brain wave characteristics, brain waves of the left frontal lobe and the right frontal lobe are asymmetric, the left frontal lobe is associated with positive thinking, and the right frontal lobe associated with negative thinking. In depression, the alpha wave increases in the left frontal lobe, the beta wave increases in the right frontal lobe, and the asymmetric become severe as symptoms become worse. A main system is composed of question monitoring, a correction module, brain wave measurement, an FFT conversion module, and a magnetic stimulation driving module.

It is possible to answer questions through the cell phone 101, and when finishing answering questions, data of an XML document format are transmitted to the server, a questioning result and a treatment history are stored in a DB, the XML document used for communication is composed of a document showing the log-in information of a doctor and a document of patient information, data is monitored in real time using the brain waves as raw data in the measurement module, maximally 250 data are received per second through 32 channels in which a high-speed data processing algorithm is used, raw data corresponding to the channels are stored in a stack for each channel, and the FFT module selects a channel and examines the frequency of a frequency by analyzing a corresponding signal.

The depression degree is found out from all the result values, and when treatment is finished, the set values of the treatment apparatus to be transmitted from the magnetic stimulation driving module to the brain wave treatment apparatus are set and stored, the set files are stored in a USB and inserted into the treatment apparatus, thereby driving and operating the treatment apparatus.

In general, the symptoms of depression appear in patients with a damaged right hemisphere rather than the left hemisphere, there is a report indicating that damage to the frontal lobe of the right hemisphere due to brain hemorrhage causes depression symptoms, but damage to the frontal lobe of the left hemisphere induces abnormally excited feeling, and depression due to damage to the brain and mental depression are too similar to discriminate therebetween. There is relevance between the alpha wave and activity, there is a study result indicating that higher activity appears in the left side of the frontal lobe, the alpha wave is more activated at the left side than the right side of the frontal lobe in a frontal lobe alpha wave asymmetry analysis study to a violent activity target, it means that the degree of activation is higher in the left frontal lobe, the functional asymmetry of the brain that activity is higher in the frontal lobe of the left hemisphere than the right hemisphere influences feeling control, and it is possible to discriminate patients without depression disorder and depression symptoms in terms of resting EEG. That is, it can be seen that the alpha wave asymmetry phenomenon in which a larger alpha wave appears at the left side than the right side of the frontal lobe is accompanied from various results related to depression. In particular, in a treatment apparatus for primates depression, it is convenient to monitor and carry, and when a current pulse of magnetic stimulation is applied to a nerve, the pulse is converted into nerve stimulation energy through stimulation and the nerve activity is promoted through nerve stimulation generated using bio nerve energy. The method having a magnetic oil type having a prevention output unit preventing damage to cranial nerves through treatment such treatment related to depression or the brain and pain attenuation may be applied to the high-tech medical field in which a coil can be replaced by attaching/detaching and superposition.

In summary, the brain is composed of the left and right hemisphere and the brain function is divided, which is called asymmetry, and primates depression increases the asymmetry of the brain, so when an abnormal symptom appears, it is possible to find out the degree of asymmetry of the left and right hemispheres through brain waves using function division.

There is a problem with the prefrontal lobe, excessive activity appears in the left frontal lobe in comparison to the right frontal lobe, and in depression, relatively low activity appears in the left frontal lobe and relatively excessive activity appears in the right frontal lobe. It can be seen that the higher the alpha wave, the lower the brain activity and the activity of the brain is relatively low in the left frontal lobe in depression. It can be seen that the higher the beta wave, the lower the brain activity and the right frontal lobe is further activated than the left frontal lobe through betadata of depression.

Accordingly, in an embodiment of the present invention, quantified brain wave unbalance data of depression are measured through EEG (electroencephalography) measurement, the data are transmitted to an IoT server platform using oneM2M standards through an Android IoT (internet of things) app, an SVM (support vector machine) model for state classification is created using brain wave measurement data and brain wave unbalance data collected in the server platform, and a depression early diagnosis and treatment apparatus is operated in accordance with the classified brain wave unbalance data.

A system that can diagnose and treat depression using brain waves measured by a high-speed data processing algorithm that can measure brain waves in wired and wireless types and can monitor the brain waves in real time is implemented, diagnosis and treatment are required through real-time connection of the cerebral cortex through synchronization of the phase of EEG by connection of a magnetic stimulation apparatus and a sensor, an EEG sensor and a stimulation coil are developed for treatment, communication is performed for accurate real-time diagnosis and treatment according to symptoms, a sensor and a stimulation coil are added, if necessary, and a cell phone can perform real-time monitoring and stimulation treatment.

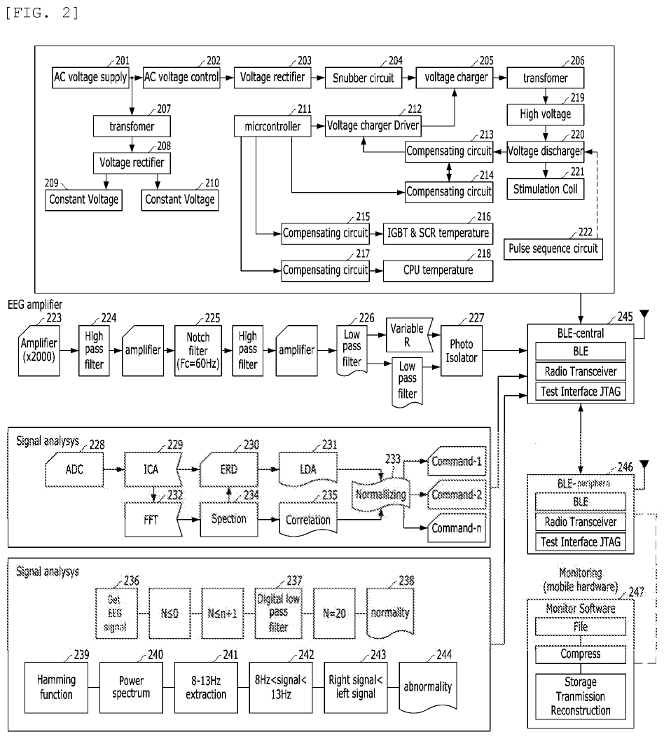

FIG. 2 is a block diagram showing a process of processing brain wave measurement data.

Reference numerals 201 to 222 denote the operation of a primates depression treatment apparatus.

First, the operation of the primates depression treatment apparatus is described. The primates depression treatment apparatus includes some or all of a power supplier 201 that supplies AC power, an AC voltage controller 202, a voltage rectifier 203, a snubber circuit 204 for power device protection, a voltage charger 205, a transformer 206, a transformer 207 for generating constant voltage, a rectifier 208 that rectifies AC voltage, a constant voltage circuit (or first constant voltage unit) 209 of DC voltage of 18V, and a constant voltage circuit (or second constant voltage unit) 210 of DC voltage of 5V. Further, the primates depression treatment apparatus includes a microprocessor 211 being in charge of main control, a voltage charge driving circuit 212, a compensation circuit 213 for a voltage discharge circuit, a compensation circuit unit 214 processing a compensation control signal for control by the microprocessor 211, an IGBT & SCR temperature compensation circuit 215, a temperature measurer (e.g., temperature sensor) 216 being in charge of the function of IGBT & SCR temperature measurement, a temperature compensator 217 performing a function of compensating for CPU temperature, a CPU temperature measurement circuit 218, a voltage converter (or high-voltage generator) 219 performing a function of increase the voltage from the transformer, a discharge 220 performing a function of discharging high voltage, a stimulation treatment coil (unit) 221, and a pulse forming network circuit 222, thereby forming necessary pulse signals in accordance with lesions.

An embodiment of the present invention uses FFT (Fast Fourier Transform) analysis that amplifies measured brain wave data, analyzes signal, analyzes signal for each abnormality measurement, expresses the signals with several sine waves or cosine waves with different amplitudes and cycles, separates the signals into a sine wave and a cosine wave, and separates waveforms into several waves in accordance with Fourier theory, and the processing method can be expressed as <Formula 1> and <Formula 2>.

$$\begin{matrix}K ( {f}_{N} ) = {\sum }_{m = 0}^{n - 1} {H}_{L} {ℓ}^{- J 2 π MN / n} = {K}_{N} & [ Formula 1 ] \\ {k}_{L} = \frac{1}{n} - {\sum }_{m = -}^{n - 1} {h}_{N}^{- J 2 π MN / n} & [ Formula 2 ]\end{matrix}$$

The aspect of a signal according to a frequency change is determined by converting a time-series signal, which changes with lapse of time, into a frequency domain, data are classified on the basis of frequency components, the density and distribution of the frequency components are found out, and indexes are applied in a recognition ability development section. The relation is as <Formula 3>.

powerratio of (snr+midb)/θ [Formula 3]

Finally, the activity of the left and right brains is applied to an AIM (Alpha Inactivity Mechanism) is applied on the basis of the theory that the brain activity is proportioned to the alpha wave, and the relations are as <Formula 4> and <Formula 5>.

$$\begin{matrix}left = \frac{1}{pactL} & [ Formula 4 ] \\ right = \frac{1}{pactR} & [ Formula 5 ]\end{matrix}$$

Reference numerals 223 to 227 indicate the configuration of an amplifier of an electroencephalography system, reference numerals 228 to 233 indicate the configuration for signal analysis, and reference numerals 236 to 244 indicate a process of analyzing an abnormal state. Reference numerals 236 to 244 may be an abnormality state analyzer.

There are provided an amplifier 223, a high-pass filter 224, a notch filter 225, a low-pass filter 226, a photo isolator 227, an A/D converter 228, an ICA (Independent Component Analysis) 229, and ERD (Event Related Desynchronization) 230, LDA (Linear discriminant analysis) 231, FFT 232, reference numeral ‘233’ indicating normality, section 234, correlation 235, an EEG signal obtaining/processing unit 236, digital low-pass filter 237, reference numeral ‘238’ indicating normality, a hamming function (unit) 239, a power spectrum (unit) 240, 8-13 Hz extraction signal detector 241, a signal analyzer 242 analyzing 8 Hz<signal <13 Hz, a brain size measurer 243 determining right signal<left signal, reference numeral ‘244’ indicating abnormality, reference numeral ‘255’ indicating Bluetooth transmitting data obtained EEG signal processing and abnormality measurement, reference numeral ‘246’ indicating Bluetooth measured and collected at a cell phone or a mobile, and reference numeral ‘247’ indicating a monitoring program that can be found out in a cell phone or mobile equipment.

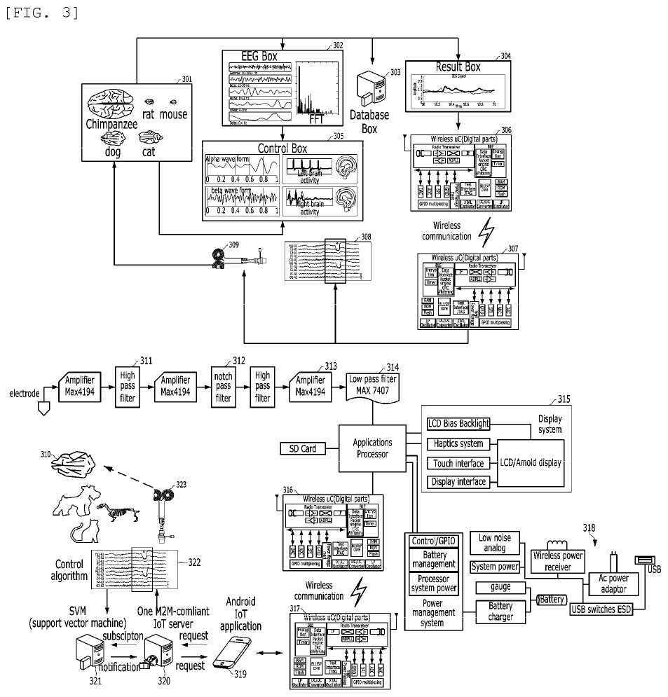

FIG. 3 is a diagram showing a primates depression treatment process according to an embodiment of the present invention.

An EEG collector, a control box, and a results viewer are fundamental components, a brain wave coming inside in real time is divided into waveforms of alpha, beta, theta, and SMR through FFT (Fast Fourier Transform) and stored in a database to construct a profile, and brain waves stored in the database are compared and calculated.

An embodiment of the present invention finds out the symmetry of the left brain and the right brain and provides it to a treatment apparatus for an alpha wave, a beta wave and asymmetry between the left brain and the right brain, and the entire system is composed of a monitoring correction module, a brain measurement module, an FFT transform module, and a primates depression treatment apparatus setting module. A PC transmits data of XML document format to a server and the server stores a treatment history in a DB.

Raw data is obtained in a brain collection measurement module, a cell phone monitors and receives data, a high-speed data processing algorithm is required, the raw data are stored in a stack, an FFT module performs FFT analysis and examination, the results are used as information for the reference of depression, the primates depression treatment apparatus sets and stores the values of the treatment apparatus to be transmitted, they are stored in a USB, etc. and inserted in the treatment apparatus, thereby performing management and treatment functions that are separately set.

Reference numeral ‘301’ indicates various brains including a primates and a mammal, reference numeral ‘302’ indicates brain waves collected from various EEG box tools, reference numeral ‘303’ indicates a database box, reference numeral ‘304’ indicates an EEG result value, reference numeral ‘305’ indicates classification for each brain wave, reference numeral ‘306’ indicates Bluetooth for transmission, reference numeral ‘307’ indicates Bluetooth for reception attached to primates depression treatment apparatus hardware, reference numeral ‘308’ indicates raw data, reference numeral ‘309’ indicates a treatment coil of the primates depression treatment apparatus, reference numeral ‘310’ indicates samples of the left and right brains of a primates, reference numerals ‘311’ to ‘313’ indicate the configuration of an EEG system, reference numeral ‘311’ indicates a high-pass filter, reference numeral ‘312’ indicates notch filter for blocking and detecting a specific frequency, reference numeral ‘313’ indicates an amplifier, and reference numeral ‘314’ indicates an application processor.

Reference numeral ‘315’ indicates a display window and a controller, reference numeral ‘316’ indicates Bluetooth attached to the microprocessor, and reference numeral ‘317’ indicates a Bluetooth attached to the cell phone. Reference numeral ‘318’ indicates portable power device for commercial power, reference numeral ‘319’ indicates a cell phone, and reference numeral ‘320’ indicates a brain wave measurement device, which measures EEG data, transmits the brain wave to an IoT server platform having oneM2M standards through an Android IoT app, and creates an SVM model of reference numeral ‘321’ and controls ASMR contents in accordance with data using the EEG data collected in the server platform. There are provided a measurement sensor, an Android IoT app, an IoT server platform, and the SVM of reference numeral ‘321’, ASMR contents control measures data from the device and transmits the data to an Android IoT app that is an open source IoT device application program entity having oneM2M standards of reference numeral ‘320’ referred to as nCube through Bluetooth communication, and the data are transmitted to an IoT server platform referred to as Mobius through an HTTP (hypertext transfer protocol). Mobius, in order to provide an IoT service on the basis of oneM2M international standards of reference numeral ‘320’, manages information of an IoT Device, controls access of the IoT device, authenticates the IoT device, manages users, provides a plurality of IoT service combination, stores the combination in a platform for serving applications and an MySQL database.

In brain wave measurement, data are transmitted to an Android IoT app (ADNAE) through Bluetooth communication, are converted into a standard forma, and are transmitted to the Android IoT app based on OneM2MAE through thing adaptation software (TAS) functioning as an interface between a S/W device and the Android IoT app. A oneM2M compatible IoT server platform registers and models the ADNAE with an infrastructure node common service entity (In CSE) for creating a container and a content instance resource. An IoT server platform (INCSE) registers, creates and uploads a resource, creates a container and a content instance resource, parses the instance resource, takes and stores uploaded EEG data into an MySQL database, and then operates the primates depression treatment apparatus when abnormality and asymmetry are severe. The depression treatment apparatus can independently operate based on data stored in a memory and can increase the treatment effect by using a database that is freely uploaded to a cell phone.

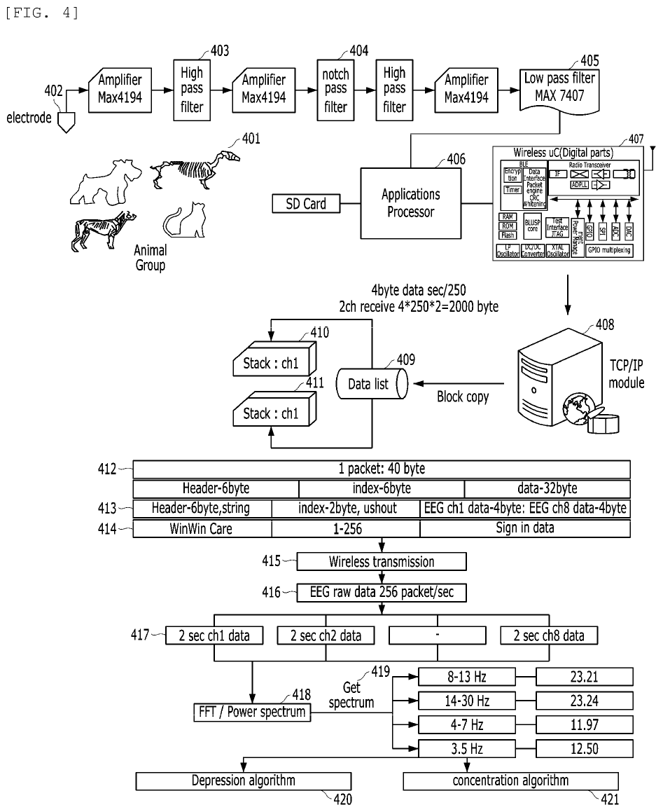

FIG. 4 is a diagram showing an analysis process of data.

Primarily, when brain measurement is finished, noise in raw data is removed through independent component analysis, the raw data are stored in a stack, signals are analyzed and examined through a channel of an FFT module, and the measurement result value is used as a reference of a depression state by calculating the degree of asymmetry of the brain. FIG. 4 shows the architecture of a high-speed processing algorithm, in which the measured brain receives 250 data per channel in 4-byte integer type, there are a total of 32 channels, and, as a result, about 3.2 KB data can be received and classified for each channel by 4 Byte×250×32 ch. There are a thread of a TCP/IP module for receiving the data and a thread processed by the measurement module, synchronization of the two threads is solved, an instance is created, and the received data of the TCP/IP module are stored and data are stored for each channel in the stack. Data collision is prevented in the TCP/IP reception module and the processing module in real time during measurement, and the data are classified for each channel and can be accurately processed when there is a call from the FFT module.

Reference numeral ‘401’ indicates several mammals, reference numeral ‘402’ indicates an electrode, reference numeral ‘403’ indicates a high-pass filter, reference numeral ‘404’ indicates a notch filter operating only at a specific frequency band, reference numeral ‘405’ indicates a low-pass filter, reference numeral ‘406’ indicates an application processor, reference numeral ‘407’ indicates a Bluetooth, reference numeral ‘408’ indicates a TCP/IP, reference numeral ‘409’ indicates a data list, reference numeral ‘410’ indicates a channel stack-1, and reference numeral ‘411’ indicates a channel stack-2.

A brain wave is measured to diagnose depression in real time, a Frontal Brain Asymmetry (FBA) ratio showing the degree of asymmetry of the frontal lobe is used, and the gap of the FBA is examined.

According to brain wave data, at channels FP1, FP2, F3, F4, F7, F8, T3, C3, C4, T4, T5, P3, P4, T6, O1, and O2, 1 packet of reference numeral ‘412’ is 40 byte, a packet of 4 byte coming through channels FP1, FP2, F3, and F4 (measurement of a depression disease) according to an embodiment of the present invention is transmitted 256 times per second of reference numeral 414 is stored at 2 seconds in real time in a system, 2 seconds of reference numeral 4132 is a minimum window size of data, and FFT analysis is used to accurately classify the brain wave into several waveforms. Reference numeral ‘414’ indicates a header, an index, and EEG ch1 data ch8 data-4 byte, reference numeral ‘414’ indicates winwin care, reference numeral ‘415’ indicates wireless transmission, reference numeral ‘416’ indicates EEG raw data 256 packet/sec, reference numeral ‘417’ indicates 2 sec ch1 data, reference numeral ‘418’ indicates an FFT/power spectrum, reference numeral ‘419’ indicates a get spectrum, reference numeral ‘420’ indicates an algorithm, and reference numeral ‘421’ indicates a concentration algorithm.

When a brain disease (depression detection index) and brain activity that are used in a data analysis process are increased, the right frontal hemisphere is associated with a negative pat, but high activity is associated with a positive part, an EEG signal is associated with the alpha wave and brain activity, the alpha wave decreases with an increase of the activity, which is inversely proportional to the alpha wave in the brain, so it is applied to an Alpha Inactivity Mechanism (AIM).

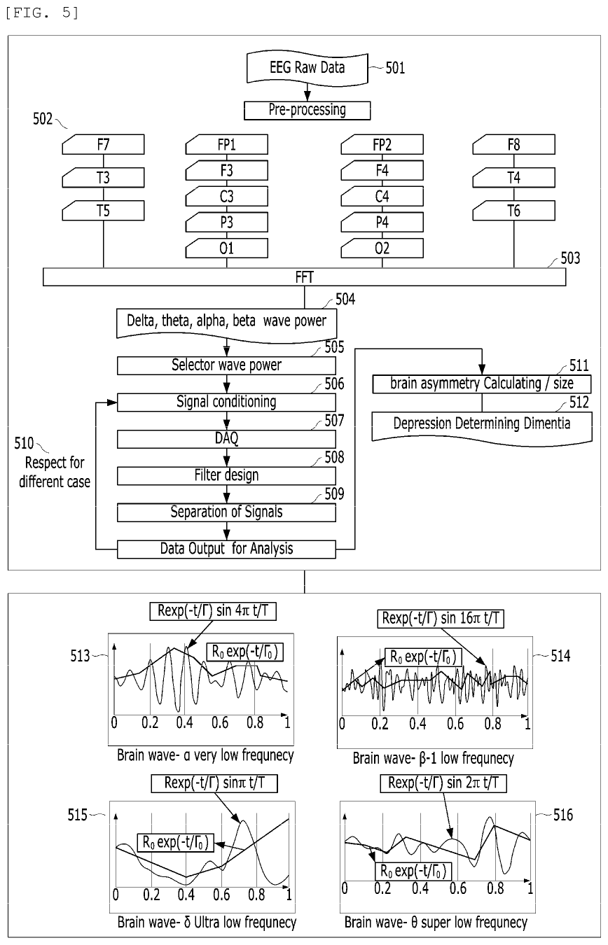

FIG. 5 is a diagram showing a driving process of an analysis algorithm according to an embodiment of the present invention.

The analysis algorithm can definitely analyze and know a depression state through two channels of the frontal lobe than six channels of the frontal lobe, and can determine a depression state through FFT after measuring the frontal lobe when the asymmetry value of the alpha wave of brain data exceeds a predetermined critical value. Reference numeral ‘501’ indicates acquisition of brain data; reference numeral ‘502’ indicates 16 arranged channels, in which a process of removing noise by applying independent component analysis to the raw data of the brain is performed, the frequency value for each channel is stored stacks for channel after the brain is processed, FFT of reference numeral ‘503’ is applied to the data stored in the stack for each channel, the frequency of a specific band such as alpha, delta, and gamma of reference numeral ‘504’ can be checked for a predetermined time from the frequency values obtained through the FFT module; reference numeral ‘506’ indicates a signal condition; reference numeral ‘507’ indicates data acquisition; reference numeral ‘508’ indicates a filter; reference numeral ‘509’ indicates returns to reference numeral ‘506’ through feedback when there is a large difference in data values analyzed through brain wave classification; and reference numeral ‘511’ indicates brain wave symmetry and a brain size, in which the degree of brain asymmetry is calculated as ±0˜values by applying an alpha wave value to the FBA algorithm in which it can be seen that the closer the vale to 0, the more symmetric the activity degree of the left and right brains, the closer the value to +1, the more the left brain is asymmetrically activated, and the closer the value to −1, the more the right brain is asymmetrically activated. Reference numeral ‘512’ can indirectly perform estimation using the entire brain wave (Alzheimer's disease, Parkinson's disease, Vascular' disease).

In the data analysis, data measured in depression is classified and analyzed, brain waveform is classified into a sine wave and a cosine wave having an amplitude and a cycle, FFT analysis that divides a wave according to Fourier series into several waves, a time-series signal that change with time is converted into a frequency domain, the signal aspect is determined, the data is classified for each frequency component, and density and distribution are found out. Finally, the activity of the left and right brains is based on the theory that activity given from a brain is inverse proportioned to the alpha wave by applying the AIM.

The difference between neuroepidemiologic coding and signal processing solves signal analysis by inducing from an entire signal set and allows for structural combination of signal objects, a neurohormone structure is required in terms of physiology, and the characteristic of an information characteristic is considered as one dimensional projection of the neuroepidemiologic codes like multiple symbol projection such as a point for 0 order projection, a line for first order projection, a square for second order projection, a cube for third order projection, and a hipercube for fourth projection. In order to convert a signal into a neuroepidemiologic signal after the signal is primarily processed, the initial information is dynamically coded, the modulation method is changed into a signal coding method, the core is to convert the initial function using a combination of 2-digit codes having parameters of a continuous pulse, reference numeral ‘513’ indicates a very low frequency, reference numeral ‘514’ indicates a low frequency, reference numeral ‘515’ indicates an ultra-low frequency, and reference numeral ‘516’ indicates a super low frequency in which Brain wave-δ, Brain wave-θ Brain wave-α, Brain wave-β are shown.

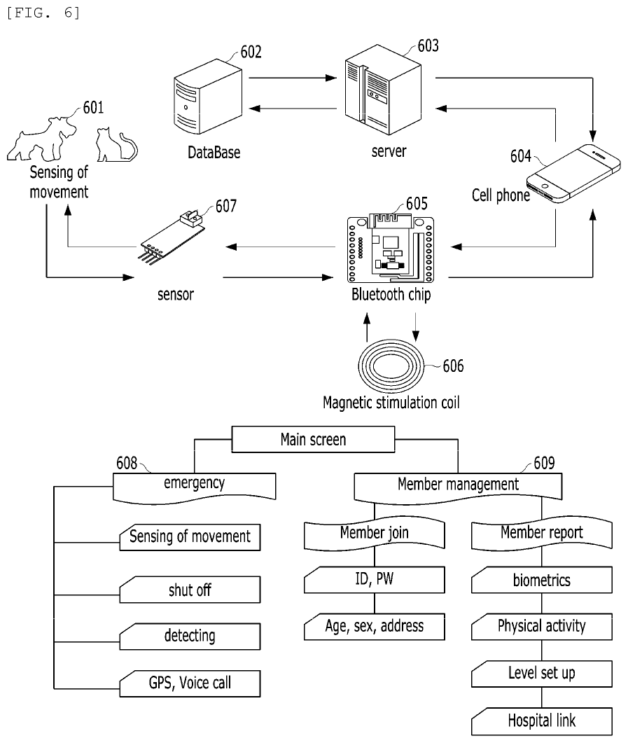

FIGS. 6 to 8 are diagrams showing a process for treatment, communication, control, and connection with an associated person.

First, in order to handle an emergency such as depression, herpes zoster, and pain treatment using a CCD-CMOS sensor and a skin and muscle conductivity sensor and manage a quick treatment performance through a cell phone, for a primates patient of reference number 601, a method according to an embodiment of the present invention reduces economic, bodily, and mental burdens and transmits database data sent and accumulated from the sensors to reference numeral 602, thereby being able to give a counsel to the patient, perform treatment, and receive special services through connection with facilities. Treatment is performed and locations are tracked using the sensors and the cell phone, and bio information and data are collected using the database, the cell phone, and the sensors and are transmitted to a medical facility through a wire/wireless system, so doctors, specialists, and guardians participate in treatment and cooperatively provide services, and uploading, feedback, disease study, and applications of a patient management system are implemented.

The apparatus proposed in an embodiment of the present invention is a system that is not limited by place and time, provides various monitoring areas, sets locations, accesses the server of reference numeral 603 through a wire/wireless communication network, and transmits a disease state and location information to a cell phone of reference numeral 604 of a guardian registered on a server together with information stored in the database when it goes out of the area. Bluetooth of reference numeral 607 is sensed by mounting the CCD-CMOS sensor and skin and muscle conductivity sensor of reference numeral 605, it is changed into an activity suitable for an emergency, and monitoring may be added to a situation that may occur by adding GPS and voice recognition functions to the cell phone.

When a disease is traced and diagnosed in the GUI (graphic user interface) type of reference numeral 608, a data system is constructed when a problem is generated in a lesion for emergency treatment, member information is stored in a database when and ID and a password are input from an external special organization, a member management monitor, and a cell phone to a server, the homepage of the external organization is accessed using Urlparse, depression, primates herpes zoster, and a communication method of a pain treatment apparatus and a cell phone through a treatment coil of reference numeral 606 that can transmit connection information with a guardian and a medical facility using a telephone number access technology is shown.

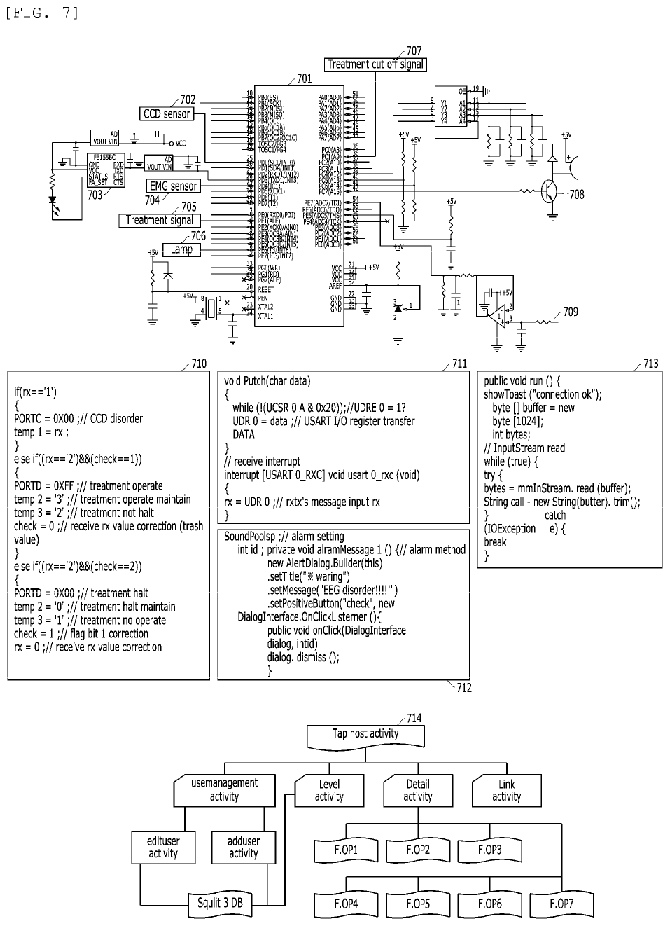

Referring to FIG. 7, according to the operation environment, a microprocessor of reference numeral 701 was operated by cooperation of a CCD-CMOS sensor of reference numeral 702, a skin, a conductivity sensor of reference numeral 4, etc., MSP430 and AVR series can be used, ATmega128 was used in the present invention, seven ports (A-G) are provided, 8 bits excluding the G port can be used, broadcast and URL that are open sources are used in response to signal of ATmega128, a homepage is liked, and a method of making an emergency phone call can be used in an unbearable emergency using ACT_CALL. According to the operation principle, when there is a problem in activity and there is no motion, LEDs are turned on, when a danger is sensed through a sensor of reference numeral 709 using an application of a cell phone, the danger is recognized, the LEDs are turned on, and when a warning signal of reference numeral 708, sensor, etc. are sensed by a cell phone application through Bluetooth of reference numeral 703, related people are informed, whereby bidirectional communication is possible through a remote operation using a web and a phone application.

In Bluetooth, when data of sensor reach a USART I/O register, a reception interrupt is called, stored in a reception message RX, and transmitted/received to/from a cell phone, a received signal of a sensor module is input to ATmega128, an input value and a signals remotely controlled by a cell phone and a sensor are converted into digital signals and stored and analyzed in the reception RX, and then output values are output.

5V and 30˜50 mA are applied to Vcc of reference numeral 703, ATmega128 is connected, 7 and 8 pins are used for transmission/reception, Status is an On/Off pin, Tx of the microprocessor is connected to the RX, a received analog signal of the sensor module is converted into a digital signal, a 1-bit character is transmitted, an ADC value of the RX is read out, a light is turned on when a problem of reference number 710 is sensed, communication of an alarm code and Bluetooth is performed in 1 bit through a USART I/O register, an interrupt is used, it is stored in a message form in the RX, a code of reference numeral 711 is transmitted and received after a problem of the sensor is sensed, the sensor module call a timer from a source code stored in Atmega128, uses a Bluetooth module for a predetermined time (Test time: 5 Min), and transmits an emergency code to the cell phone, reference numeral 712 sends an emergency alarming code to the cell phone, an application informs reference numeral 713 of the problem with the sensor, and primates depression, primates herpes zoster, and pain treatment apparatus of reference numeral 705 and 707 is operated to performed treatment and then stopped.

When a signal of Bluetooth is received, it is changed into activity that is an execution unit configuring a screen, an interface is implemented, a server socket is generated, a Bluetooth search is executed when a connection request is sensed, whether there is connected pairing, a client that is an important part is connected, a socket is generated, transmission/reception is performed through the socket, and a listening socket is closed and a thread is ended when socket generation fails.

Next, an input/output stream is set, a UUID protocol is used, a code configuring an environment that can communicate with Bluetooth is set, activity configuration is developed using Java and Xml in an android platform in application design, the OS of a cell phone uses Android, an application production method uses Eclipse and JDK, the version is 2.3.3 gingerbread, the development environment is a Jetty server and an Elasticsearch database system, and a DB uses Elastic Search and database SQLite having Android as a search engine.

According to the application structure, reference numeral 714 for five services of a CCD sensor, muscle conductor, operation sensing, GPS, and emergency phone call that are performed after a signal is transmitted to a cell phone through Bluetooth, and diagnosis and management information is configured by eight activities, there is an activity configuration in the previous page, it is implemented in a server in HostActivity, log-in and a member information storage method are implemented in UserManagement, Tomcat that is an existing server requires many settings, a simple Jetty built-in server and Java language are used, and cell phone application is not operated when an emergency application requested by a client insets a corresponding right to an Manifast.xml file in an actual operation and then does not designate the right.

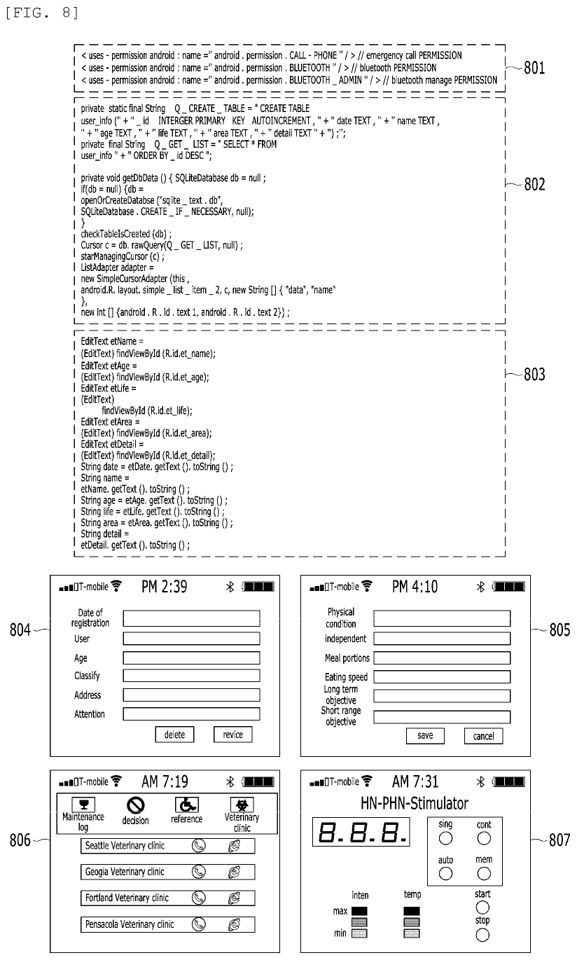

Referring to FIG. 8, for creating a database, a use log-in button and a member jointing button are made using an Oncreate method of reference numeral 801, EditText such as an ID, a password, an age, an address, a house type, a daily life, and life information is made, repetition of IID and password information is checked using a SetOnClicklistener method, a DB database of reference numeral 802 is created, a code configures a table, a table User info is created, user IDs are automatically increased, a test type key value is added, a table value and a key value are received from Sqlite test, and a reference numeral 803 receives a list of Key values from EditText as a code in a process of receiving and updating data in a table created by receiving user input, stores it as a string value, and then stores it in a DB file.

According to a result of implementing a mobile application, life and healthcare information are collected and recognized with concerned persons and guardian for early diagnosis and treatment through reference numeral 804, and can be referred to for external evaluation indexes such as neurology, BPSD, daily life activity, and performance ability, information can be supported from external instructions, disease progression is continuously monitored, and it is possible to safely cope with an emergency when an emergency occurs. Further, when a user name, a house type, a name, an age, and a dwelling area are input and stored in an image implemented by data collection and support by a diagnosis support function, a diagnosis history image of an external medical organization shows up, and weekly and monthly collected data are data to be used for diagnosis and treatment management, whereby it is possible to verify diagnosis of depression, herpes zoster, and zoster post-pain.

According to the image implemented in reference numeral 805, family members or guardians can periodically input symptoms and performance abilities that are shown in daily life, and can evaluate activities, mental, meals, excretion, bathing, wearing clothes, cleanness, etc. in levels (1˜5) on the basis of observed results, diagnosis evaluation is downloaded as an external index and referred to for level determination when observation input such as eating activity, the amount of a meal, a speed, and the degree of independence is finished, and information about alternative mediation treatment such as treatment, medicinal treatment, correction support, and whether facility program support is provided is provided through short and long-term objects. Further, according to an image of a connected treatment support of reference numeral 806, when in-house treatment is difficult in accordance with a management support function and a level determination result by observation, it is possible to provide a help for smooth treatment by connecting patient information to a close primates hospital or a special clinic, and appropriate mediation and connected treatment based of determination by a behavior and BPSD doctor are proposed. Reference numeral 807 performs early treatment by setting treatment in accordance with diagnosis and situations through communication between a cell phone and an apparatus.

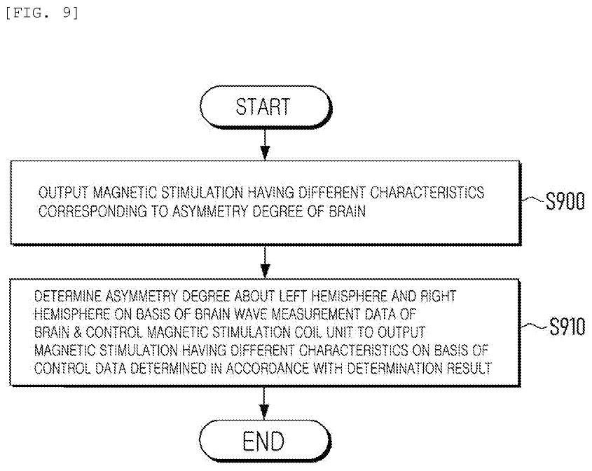

FIG. 9 is a flowchart showing a driving process of the primates depression treatment apparatus of FIG. 1 according to another embodiment of the present invention.

Referring to FIG. 9 with FIG. 1 for the convenience of description, the primates depression treatment apparatus 90 of FIG. 1 according to another embodiment of the present invention outputs magnetic stimulation having different characteristics corresponding to an asymmetry degree of a brain (S900). The asymmetry degree of the brain shows the asymmetry degree of the left hemisphere (or left brain) and the right hemisphere (right brain), and the characteristic of magnetic stimulation, that is, the frequency, the intensity of stimulation, etc. may depend on the degree.

To this end, the primates depression treatment apparatus 90 determines the asymmetry degree about the left hemisphere and the right hemisphere on the basis of brain wave measurement data of the brain and controls the magnetic stimulation coil unit (or depression treatment unit) 125˜139 to output magnetic stimulation having different characteristics on the basis of control data determined in accordance with the determination result.

Although an EEG sensor, etc. are used for brain wave measurement in an embodiment of the present invention, it is possible to determine the asymmetry degree of the brain in various ways, so it is possible to determine the asymmetry degree of the brain using an imaging technique. In this case, a method different from those described above may be applied, but it is not that much different in respect of varying the treatment stimulation in accordance with the asymmetry degree of the brain.

Other than the above description, the primates depression treatment apparatus 90 can perform various operations, and other details were sufficiently described above and are no longer described.

Even through all components of embodiments of the present invention are combined in one unit or operated in combination in the above description, the present invention is not limited thereto. That is, the all components may be selectively combined and operated within the scope of the present invention. Further, although all the components may be implemented as individual hardware, some or all of the components may be selectively combined as computer programs having program modules that perform some or all of functions combined in one or several items of hardware. Codes and code segments of the computer programs may be easily inferred by those skilled in the art. The computer programs may be stored in a nontransitory computer readable media and read out and executed by a computer, thereby achieving embodiments of the present invention.

The nontransitory computer readable media is not a media that stores data for a short time such as a register, a cache, and a memory, but a media that can semipermanently store data and can be read out by a device. In detail, the programs may be stored and provided in a nontransitory computer readable media such as a CD, a DVD, a hard disk, a Blu-ray disc, a USB, a memory card, and a ROM.

Although exemplary embodiments of the present disclosure were illustrated and described above, the present disclosure is not limited to the specific exemplary embodiments and may be modified in various ways by those skilled in the art without departing from the scope of the present disclosure described in claims, and the modified examples should not be construed independently from the spirit of the scope of the present disclosure.

What is claimed is:

1. A primates depression treatment apparatus comprising:
a magnetic stimulation coil unit outputting magnetic stimulation having different characteristics corresponding to an asymmetry degree of a brain; and
a controller determining an asymmetry degree between a left hemisphere and a right hemisphere on the basis of brain wave measurement data of the brain, and controlling the magnetic stimulation coil unit to output the magnetic stimulation having difference characteristics on the basis of control data that are determined in accordance with the determination result.

2. The primates depression treatment apparatus of claim 1, wherein the controller creates quantified brain wave imbalance data of depression on the basis of the brain wave measurement data, classifies the created brain wave imbalance data in accordance with a state of the brain, and outputs the control data on the basis of the classified brain wave imbalance data.

3. The primates depression treatment apparatus of claim 2, wherein the controller performs classification according to the state of the brain on the basis of a first analysis result of an α-wave, a β-wave, a γ-wave, a δ-wave, and a θ-wave on the basis of the brain wave measurement data and a second analysis result about sizes of the left hemisphere and the right hemisphere.

4. The primates depression treatment apparatus of claim 3, wherein the controller applies a Support Vector Machine (SVM) model to classify the brain imbalance data according to the state of the brain.

5. The primates depression treatment apparatus of claim 1, wherein the controller performs wired or wireless communication for providing the control data to the magnetic stimulation coil unit when the controller is disposed in a device separated from the magnetic stimulation coil unit.

6. A method of driving a primates depression treatment apparatus comprising:
outputting magnetic stimulation having different characteristics corresponding to an asymmetry degree of a brain; and
determining an asymmetry degree between a left hemisphere and a right hemisphere on the basis of brain wave measurement data of the brain, and controlling the magnetic stimulation coil unit to output the magnetic stimulation having difference characteristics on the basis of control data that is determined in accordance with the determination result, by means of a controller.

7. The method of claim 6, further comprising:
creating quantified brain wave imbalance data of depression on the basis of the brain wave measurement data;
classifying the created brain wave imbalance data in accordance with a state of the brain; and
outputting the control data on the basis of the classified brain wave imbalance data.

8. The method of claim 7, wherein the classifying according to a state of the brain performs classification according to the state of the brain on the basis of a first analysis result of an α-wave, a β-wave, a γ-wave, a δ-wave, and a θ-wave on the basis of the brain wave measurement data and a second analysis result about sizes of the left hemisphere and the right hemisphere.

9. The method of claim 8, wherein the classifying according to a state of the brain applies an SVM model to classify brain wave imbalance data according to the state of the brain.

10. The method of claim 6, further comprising performing wired or wireless communication for providing the control data to the magnetic stimulation coil unit, in a device separated from the magnetic stimulation coil unit.
